SEQUENCE LISTING (7 sequences)

SEQ ID NO: 1 - protein, 13 aa, Artificial Sequence
  CLOCK and/or PER1 gene activator
  misc_feature (1)..(4): Xaa can be any naturally occurring amino acid or no amino acid
  misc_feature (5): Xaa can be threonine or serine or no amino acid
  misc_feature (9): Xaa can be isoleucine, leucine, proline, valine, alanine, glycine or no amino acid
  misc_feature (10)..(13): Xaa can be any naturally occurring amino acid or no amino acid
XXXXXSTPXXXXX

SEQ ID NO: 2 - protein, 7 aa, Artificial Sequence
  CLOCK and/or PER1 gene activator
YVSTPYN

SEQ ID NO: 3 - protein, 5 aa, Artificial Sequence
  CLOCK and/or PER1 gene activator
VSTPE

SEQ ID NO: 4 - protein, 6 aa, Artificial Sequence
  CLOCK and/or PER1 gene activator
LHSTPP

SEQ ID NO: 5 - protein, 6 aa, Artificial Sequence
  CLOCK and/or PER1 gene activator
RHSTPE

SEQ ID NO: 6 - protein, 5 aa, Artificial Sequence
  CLOCK and/or PER1 gene activator
HSTPE

SEQ ID NO: 7 - protein, 4 aa, Artificial Sequence
  CLOCK and/or PER1 gene activator
SPLQ

(19) United States
(12) Patent Application Publication
(10) Pub. No.: US 2018/0125778 A1
(43) Pub. Date: May 10, 2018

(54) Topical Compositions And Methods For Stimulating MIR-146A In Skin Cells

(71) Applicant: ELC Management LLC, Melville, NY (US)

(72) Inventors: Nadine Pernodet, Huntington Station, NY (US); Klodjan Stafa, Melville, NY (US); Edward Pelle, Valley Stream, NY (US); Kelly Dong, Merrick, NY (US); Earl Goyarts, Commack, NY (US); Dawn Layman, Ridge, NY (US); Krystle Corallo, Huntington, NY (US)

(21) Appl. No.: 15/794,672

(22) Filed: Oct. 26, 2017

Related U.S. Application Data
(60) Provisional application No. 62/419,661, filed on Nov. 9, 2016.

(51) Int. Cl. A61K 9/00; C12N 15/113; A61K 35/745; A61K 36/185; A61K 38/06; A61K 38/43

(52) U.S. Cl. CPC A61K 9/0014; C12N 15/113; A61K 35/745; C12N 2320/31; A61K 38/06; A61K 38/43; C12N 2310/141; A61K 36/185

(57) ABSTRACT

Topical compositions containing at least one ingredient that stimulates Mir-146a activity in skin cells, methods for treating skin and methods for screening for ingredients that stimulate Mir-146a activity.

### TECHNICAL FIELD

The invention is in the field of topical compositions and methods for stimulating MicroRNA 146a (“Mir-146a) expression in skin cells and methods for screening for actives that stimulate Mir-146a for formulation into topical compositions to treat skin for improvement.

### BACKGROUND OF THE INVENTION

Micro RNAs (MiRNA) are regulatory molecules that affect all aspects of cellular biology. MiRNAs are small non-coding molecules of about 20-25 nucleotides (nt) that are involved in post-transcriptional gene regulation. They are regulatory molecules that influence all aspects of cellular biology including immune response. Mir-146a in particular has been shown to be an important regulator of innate and adaptive cellular immunity. It has been shown that aberrations in Mir-146a are found in various disease states such as cancer, thyroid dysfunction, and hematopoietic disorders.

We have shown that Mir-146a levels decrease with age and that the diminution of Mir-146a has a direct impact on the cellular PER1 gene. PER1 refers to Period Homolog gene, which also influences circadian activity. With diminished cellular Mir-146a and PER1 levels, night cellular synchronization is compromised. This in turn minimizes the repair activities that take place during nightly rest. We have also shown that Mir-146a activity significantly compromises DNA repair resulting in increased cellular DNA damage. Thus ingredients that activate Mir-146a will positively impact PER1 activity, DNA repair, and promote stem cell health (also referred to as sternness).

The invention is directed to topical compositions and methods for stimulating Mir-146a expression in skin cells as well as a method for screening for ingredients that stimulate Mir-146a expression in skin cells which will in turn promote PER1 gene expression, DNA repair, and stemness.

### SUMMARY OF THE INVENTION

The invention is also directed to topical compositions containing at least one ingredient that stimulates Mir-146a expression in skin cells in combination with an adjuvant.

The invention is also directed to method for stimulating, in skin cells: (a) Mir-146a expression, and (b) CLOCK or PER1 gene expression, and/or (c) cellular DNA repair; and/or (d) stemness by topically applying a composition containing an adjuvant and one or more ingredients that provide effects (a), (b), (c), and (d).

The invention is also directed to a method for treating skin to restore the natural temporal rhythm of skin to promote healthier skin by applying a topical composition comprising an adjuvant and at least one ingredient that stimulates Mir-146a expression in skin cells. The improvements may be in one or more benefits selected from the group: (a) moisturization, (b) reducing skin redness or inflammation, (c) minimizing the appearance of lines and wrinkles, (d) evening skin tone, (e) minimizing the appearance of irregularities and age spots on hands, face, and neck, (f) improving the appearance of uneven pigmentation, (g) repairing skin that is damaged by exposure to sunlight, pollution, environmental conditions, and the like, (h) improving skin laxity, or combinations thereof, by topically applying a composition that contains one or more ingredients that: (i) stimulate Mir-146a expression, and (ii) improve repair of damaged cellular DNA, and/or (iii) stimulate CLOCK or PER1 gene activity, and/or (iv) promote stemness by maintaining expression of stem cell markers that are inherent in stem cells.

The invention is directed to a method for formulating a topical composition that contains an ingredient that stimulates expression of Mir-146a activity in skin cells comprising the steps of:

(a) treating skin cells in vitro with an ingredient,

(b) extracting RNA from skin cells,

(c) reverse transcribing RNA to form cDNA strands,

(d) amplifying and quantifying the cDNA strands complementary to Mir-146a by probing with a primer sequence and annealing cDNA strands complementary to Mir-146a,

(e) selecting the ingredient that shows an increase in cDNA complementary to Mir-146a when compared to untreated cells.

### DESCRIPTIONS OF THE DRAWINGS

FIG. 1 graphically demonstrates the increase in Mir-146a expression in skin cells treated with Baobab, or Adansonia digitata extract.

FIGS. 2A, 2B and 2C demonstrate the increase in Mir-146a expression in dermal fibroblasts from 62 and 42 year old donors when treated with Porphyridium cruentum red algae extract, Polysea.

FIGS. 3A, 3B and 3C demonstrate that when dermal fibroblasts were treated with Moringa oleifera extract no increase in expression of Mir-146a was seen.

FIGS. 4A. 4B and 4C demonstrate that when dermal fibroblasts were treated with sesame oil no increase in Mir-146a expression was seen.

FIG. 5 demonstrates the relationship between Mir-146a activation and PER1 expression by showing that when Mir-146a expression is inhibited, PER1 expression is correspondingly reduced.

FIG. 6 demonstrates the effects of Mir-146a inhibition and PER1 inhibition on dermal fibroblasts from donors of different ages and shows that the difference increases with increasing age of dermal fibroblasts.

FIG. 7 demonstrates the direct correlation between Mir-146a activity and PER1 activity. In particular, stimulating Mir-146a causes activation of PER1 and vice versa.

FIG. 8 shows that Mir-146a activation decreases with age.

FIG. 9 demonstrates that stimulation of Mir-146a promotes DNA repair in cells.

### DETAILED DESCRIPTION

### The Topical Composition

### I. The Mir-146a Activator

The composition of the invention contains at least one ingredient that stimulates Mir-146a expression in skin cells. The Mir-146a activator may be present in amounts ranging from about 0.001 to 10%, preferably from about 0.005 to 3%, more preferably from about 0.01 to 2% with all percentages by weight of the total composition.

In one embodiment the ingredient is a botanical extract that is obtained by aqueous extraction of Baobab according to a method set forth in U.S. Patent Application No. 2003/0092168 which is hereby incorporated by reference in its entirety. Such a method involves extracting, in aqueous medium, the plant material at a pH of 9 to 13; separating the plant material and reducing the pH of the mixture to 5 to 8, then recovering the aqueous extract; and purifying the extract by removing insoluble material. Alternatively, the pH reduction can occur before or after the enzymatic treatment. A suitable cellulolytic material includes cellulases or pectinases. Preferred treatment temperature may range from 20 to 70° C. and may take from ½ to 2 hours. The separation of the extract from the crude plant material may be done using centrifuges, filters, or screens. Most preferred is where the plant material is first contacted with an aqueous solution having a pH of 9 to 13 at a temperature of 20 to 70° C. for 1 to 2 hours. The crude aqueous extract is separated from the plant material with a centrifuge or filter, then treated with a cellulolytic enzyme (preferably pectinase or cellulase) at a pH of 5 to 8 under similar temperature and time conditions used for the first extraction. The insoluble material is separated off again to obtain a purified aqueous extract that may be freeze dried or spray dried. This method ensures that the plant material in the aqueous extract contains a significant amount of RNA, in fact up to about 25%, or from about 0.1 to 5%, preferably from about 0.2 to 2% by weight of the total extract.

One particularly preferred Baobab extract is sold by Ashland Specialty Ingredients under the trade name Mirage 146a™ which has the INCI name Adansonia digitata extract in a mixture along with glycerin and water. Baobab extract is obtained from the African Baobab tree. Baobab tissue is unique because it contains 4 chromosomes in each set instead of 2 and the tree itself does not exhibit growth rings typically found in hardwood trees. This Baobab contains small RNAs which may be obtained by extraction from the seeds of the Baobab tree.

Examples of other ingredients that stimulate Mir-146a include Acorus calamus extract and Algae extract from Porphyridium cruentum red microalgae. Such algae extract is manufactured according to a process set forth in U.S. Pat. No. 5,534,417 which is hereby incorporated by reference in its entirety.

The selected ingredient may be formulated into a variety of topical products such as skin creams, lotions, serums, sprays, gels, solutions, or suspensions. The ingredient may be incorporated into the composition in amounts ranging from about 0.01 to 10%, preferably from 0.05 to 5%, more preferably from about 0.1 to 3% with all percentages mentioned herein by weight.

Most preferred is where the topical compositions are in the form of emulsions, either water-in-oil or oil-in-water. Most preferred are oil in water emulsions containing from about 10-95% water and 5-45% oil including other adjuvants including, but not limited to, those set forth below.

The term “adjuvant” means an ingredient that increases the efficacy of the composition into which the Mir-146a activator is formulated without compromising its activity, specifically: DNA repair enyzmes, PER1 or CLOCK gene expression activators, autophagy activators, proteasome activators, and probiotic microorganisms from Bifidobacterium or Lactobacillus genus.

### II. Autophagy Activators

Autophagy activators may be suitable adjuvants. It is desirable for the composition to contain, in addition to the ingredient that stimulates Mir-146a expression at least one ingredient that activates normal cellular autophagic processes. The autophagy activator is present in amounts ranging from about 0.00001 to 20%, preferably 0.0001-5%, more preferably from about 0.001 to 1%. In general, the cellular autophagy process comprises four general steps. Step 1 is the initiation of vacuole formation; Step 2 the formation of the initial vacuole or autophagosome which sequesters the cytoplasmic material to be degraded. Step 3 is the maturation of the autophagosome into a degradative vacuole. Step 4 is the actual degradation of the sequestered material.

Ingredients with autophagy activation activity can be identified by their ability to either stimulate or inhibit various cellular metabolic pathways. For example, ingredients that stimulate the expression of MAP-LC3, ATG5-12, protein p53, AMPK, or DRAM are suitable autophagy activators. Ingredients that inhibit the expression of mTOR are also suitable autophagy activators.

The gene MAP-LC3 codes for microtubule-associated protein 1 light chain 3, a protein that initiates formation of autophagosomes. ATG5-12 also stimulates formation of autophagosomes. mTOR, also known as mammalian target of rapamycin, is also known as the mechanistic target of rapamycin or FK506 binding protein 12-rapamycin associated protein 1 (FRAP1). FRAP1 is encoded by the FRAP gene. Any ingredient that inhibits the expression of mTOR, involved in autophagosome creation, will have autophagy activating properties. Also suitable as autophagy activators are ingredients that stimulate expression of protein p53, AMPK, and/or DRAM (damage remedy autophagy modulator protein) in keratinocytes. Protein p53, also known as a tumor suppressor protein, is encoded by the p53 gene. AMPK means AMP activated protein kinase and DRAM, damage related autophagy modulator. Both are known to stimulate autophagy activation in keratinocytes.

Thus any ingredient that has the above mentioned effects on the genes may be suitable autophagy activators. During the autophagocytic process cellular debris such as oxidized proteins and peroxidized lipids are degraded. Such cellular debris often affects normal metabolic function. Screening of ingredients to determine efficacy by ability to stimulate or inhibit cellular, preferably keratinocyte, genes and/or proteins mentioned above may be done according to methods as set forth in US Patent Publication No. 2011/0243983 or other methods known in the art.

For example, one general process for identifying ingredients that may be autophagy activators is by first inducing nutritive stress in cultured cells such as keratinocytes. For example, the cells are first cultured in complete culture medium with growth factors, for about 24 hours. The culture medium is then removed and replaced with a non-nutritive culture medium, for example one that does not contain growth factors. The cells are cultured for about 30 minutes to about 25 hours in a state of nutritive stress. Then, the non-nutritive culture medium is removed and replaced with complete culture medium to promote cellular recovery. Thereafter, the cells are evaluated for autophagocytic activity by measuring the expression of one or more of MAP-LC3; ATGS-12; phosphorylated mTOR; phosphorylated p53; DRAM; or phosphorylated AMPK in those cells. Measurement of such expression can take place by immunofluorescence measurements. In addition, the expression can be ascertained by Western Blot analysis of phosphorylated proteins associated with the expressed genes.

Examples of ingredients that are known to exert either the stimulatory or inhibitory effects on the above mentioned genes which, in turn, stimulate autophagy, are yeast extracts including but not limited to those from the genuses such as Lithothamnium, Melilot, Citrus, Candida, Lens, Urtica, Carambola, Momordica, Yarrowia, Plumbago, etc. Further specific examples include Lithothamniumn calcareum, Melilotus officinalis, Citrus limonum, Candida saitoana, Lens culinaria, Urtica dioica, Averrhoa carambola, Momordica charantia, Yarrowia lipolytica, Plumbago zeylanica and so on.

Also suitable are ingredients such as amiodarone hydrochloride, GF 109203X which is also referred to as (3-(N-[Dimethylamino]propyl-3-indolyl)-4-(3-indolyl)maleimide 3-[1-[3-(Dimethylamino)propyl]1H-indol-3-yl]-4-(1Hindol-3-yl)1H-pyrrole-2,5dione Bisindolylmaleimide I; N-Hexanoyl-D-sphingosine; Niclosamide; Rapamycin from Streptomyces hygroscopicus; Rottlerin which is also referred to as (1-[6-[(3-Acetyl-2,4,6-trihydroxy-5-methylphenyl)methyl]-5,7-dihydroxy-2,2-dimethyl-2H-1-benzopyran-8-yl]-3-phenyl-2-propen-1-one, Mallotoxin); STF-62247, also known as 5-Pyridin-4-yl-thiazol-2-yl-m-tolyl-amine; Tamoxifen; Temsirolimus which is also known as 42-[3-Hydroxy-2-methylpropanoate, CCI-779, Rapamycin; ATG1 autophagy related 1 homolog; ATG1, Serine/threonine-protein kinase ULK1, UNC-51-like kinase; or Z36 which is also referred to as ((Z)-5-Fluoro-1-(3′-dimethylamino)propyl-3-[(5′-methoxyindol-3-ylidene)methyl]-indolin-2-one; or 1-[3-(dimethylamino)propyl]-5-fluoro-1,3-dihydro-3-[(5-methoxy-1H-indol-3-yl)methylene]-2H-Indol-2-one); Bufalin, also referred to as 3β,14-Dihydroxy-5β,20(22)-bufadienolide, 5β,20(22)-Bufadienolide-3β,14-diol. Such ingredients may be purchased from Sigma-Aldrich Chemical Company.

### III. Proteasome Activator

It is desirable for the composition to contain an adjuvant that stimulates proteasome activity in skin cells. If present the proteasome activator may range from 0.0001 to 35%, preferably from about 0.0005 to 15%, more preferably from about 0.001 to 5%.

Suitable proteasome activators are any compounds, molecules, or active ingredients that stimulate proteasome activity in the cells of keratin surfaces.

Examples of suitable proteasome activators include, but are not limited to, algin, alginates, hydrolyzed algin, molasses extract, Trametes extracts, including extracts from Trametes versicolor, olea hydroxol.

### IV. CLOCK, PER1 Gene Activator

CLOCK or PER1 cellular gene activators are also suitable adjuvants. Suggested ranges are from about 0.000001 to about 5%, preferably from about 0.000005 to 3.5%, more preferably from about 0.00001 to 2%. Suitable CLOCK or PER1 activators may be present in the form of botanical extracts, polypeptides, peptides, amino acids, and the like.

A. Peptide CLOCK or PER1 Gene Activators

A particularly preferred CLOCK and/or PER1 gene activator comprises a peptide of the formula (I):

R₁-(AA)ₙ-X₁—S-T-P—X₂-(AA)ₚ-R₂

where
(AA)ₙ-X₁—S-T-P—X₂-(AA)ₚ is (SEQ ID No. 1), and:
X₁ represents a threonine, a serine, or no amino acid,
X₂ represents an isoleucine, leucine, proline, valine, alanine, glycine, or no amino acid,
AA represents any amino acid or derivative thereof, and n and p are whole numbers between 0 and 4,
R₁ represents the primary amine function of the N-terminal amino acid, either free or substituted by a protective grouping that may be chosen from either an acetyl group, a benzoyl group, a tosyl group, or a benzyloxycarbonyl group,
R2 represents the hydroxyl group of the carboxyl function of the C-terminal amino acid, substituted by a protective grouping that may be chosen from either a C1 to C20 alkyl chain or an NH2, NHY, or NYY group with Y representing a C1 to C4 alkyl chain,
   wherein the sequence of general formula (I) comprises from about 3 to 13 amino acid residues, said sequence of general formula (I) possibly containing substitutions of amino acids X₁ and X₂ with other chemically equivalent amino acids; wherein the amino acids are: Alanine (A), Arginine (R), Asparagine (N), Aspartic Acid (D), Cysteine (C), Glutamic Acid (E), Glutamine (Q), Glycine (G), Histidine (H), Isoleucine (I), Leucine (L), Lysine (K), Methionine (M), Phenylalanine (F), Proline (P), Serine (S), Threonine (T), Tryptophan (W), Tyrosine (Y), Valine (V). More preferred, are peptides of the above formula, as follows:

S-T-P-NH₂
Ser-Thr-Pro-NH₂
(SEQ ID No. 2)
Y-V-S-T-P-Y-N-NH₂
Tyr-Val-Ser-Thr-Pro-Tyr-Asn-NH₂
(SEQ ID NO. 3)
NH₂-V-S-T-P-E-NH₂
NH₂-Val-Ser-Thr-Pro-Glu-NH₂
(SEQ ID No. 4)
NH₂-L-H-S-T-P-P-NH₂
NH₂-Leu-His-Ser-Thr-Pro-Pro-NH₂
(SEQ ID No. 5)
CH₃NH-R-H-S-T-P-E-NH₂
CH₃-NH-Arg-His-Ser-Thr-Pro-Glu-NH₂
(SEQ ID No. 6)
CH₃NH-H-S-T-P-E-CH₃NH
CH₃-NH-His-Ser-Thr-Pro-Glu-CH₃-NH

More preferred is the S-T-P—NH₂ peptide, SEQ ID No. 4, or mixtures thereof. Most preferred is a peptide manufactured by Ashland under the trademark Chronolux® having the INCI name Tripeptide-32.

Another preferred CLOCK and/or PER1 gene activator is manufactured by Ashland under the trademark Chronogen® having the INCI name Tetrapeptide-26. The amino acid sequence for Tetrapeptide-26 is

(SEQ ID No. 7)
S-P-L-Q-NH₂
Ser-Pro-Leu-Gln-NH₂.

B. Botanical Extracts

Also suitable as the CLOCK or PER1 gene activator is cichoric acid or isomers or derivatives thereof. Cichoric acid may be synthetic or naturally derived. Synthetic cichoric acid may be purchased from a number of commercial manufacturers including Sigma Aldrich. Cichoric acid may also be extracted from botanical sources that are known to contain cichoric acid such as Echinacea, Cichorium, Taraxacum, Ocimum, Melissa, or from algae or sea grasses. More specifically, botanical extracts such as Echinacea purpurea, Cichorium intybus, Taraxacum officinale, Ocimum basilicum, or Melissa officinalis. The term “cichoric acid” when used herein also includes any isomers thereof that are operable to increase PER1 gene expression in skin cells.

A specific example includes a botanical extract from Echinacea purpurea sold by Symrise under the brand name Symfinity™ 1298 which is an extract of Echinacea purpurea which is standardized during the extraction process to contain about 3% by weight of the total extract composition of cichoric acid. Echinacea extracts from different sources will vary in cichoric acid content, and as such will yield variable results in induction of PER1 gene expression. Ethanolic extract of the roots of Echinacea purpura will provide more cichoric acid than ethanolic extracts of Echineacea augustifolia or Echinacea pallida. The content of active ingredients in any extract is also very dependent on the method of extraction. For example, it is known that in many cases enzymatic browning during the extraction process will reduce the phenolic acid content of the resulting extract.

### V. DNA Repair Enzymes

The composition used in the method of the invention may also contain one or more DNA repair enzymes as adjuvants. Suggested ranges are from about 0.00001 to about 5%, preferably from about 0.00005 to about 3%, more preferably from about 0.0001 to about 2% of one or more DNA repair enzymes.

DNA repair enzymes as disclosed in U.S. Pat. Nos. 5,077,211; 5,190,762; 5,272,079; and 5,296,231, all of which are hereby incorporated by reference in their entirety, are suitable for use in the compositions and method of the invention. One example of such a DNA repair enzyme may be purchased from AGI/Dermatics under the trade name Roxisomes®, and has the INCI name Arabidopsis Thaliana extract. It may be present alone or in admixture with lecithin and water. This DNA repair enzyme is known to be effective in repairing 8-oxo-Guanine base damage.

Another type of DNA repair enzyme that may be used is one that is known to be effective in repairing 06-methyl guanine base damage. It is sold by AGI/Dermatics under the tradename Adasomes®, and has the INCI name Lactobacillus ferment, which may be added to the composition of the invention by itself or in admixture with lecithin and water.

Another type of DNA repair enzyme that may be used is one that is known to be effective in repairing T-T dimers. The enzymes are present in mixtures of biological or botanical materials. Examples of such ingredients are sold by AGI/Dermatics under the tradenames Ultrasomes® or Photosomes®. Ultrasomes® comprises a mixture of Micrococcus lysate (an end product of the controlled lysis of various species of micrococcus), lecithin, and water. Photosomes® comprise a mixture of plankton extract (which is the extract of marine biomass which includes one or more of the following organisms: thalassoplankton, green micro-algae, diatoms, greenish-blue and nitrogen-fixing seaweed), water, and lecithin.

Another type of DNA repair enzyme may be a component of various inactivated bacterial lysates such as Bifida lysate or Bifida ferment lysate, the latter a lysate from Bifido bacteria which contains the metabolic products and cytoplasmic fractions when Bifido bacteria are cultured, inactivated and then disintegrated. This material has the INCI name Bifida Ferment Lysate.

Other suitable DNA repair enzymes include Endonuclease V, which may be produced by the denV gene of the bacteriophage T4. Also suitable are T4 endonuclease; O⁶-methylguanine-DNA methyltransferases; photolyases such as uracil- and hypoxanthine-DNA glycosylases; apyrimidinic/apurinic endonucleases; DNA exonucleases, damaged-bases glycosylases (e.g., 3-methyladenine-DNA glycosylase); correndonucleases either alone or in complexes (e.g., E. coli uvrA/uvrB/uvrC endonuclease complex); APEX nuclease, which is a multi-functional DNA repair enzyme often referred to as “APE”; dihydrofolate reductase; terminal transferase; topoisomerase; O⁶ benzyl guanine; DNA glycosylases.

Other types of suitable DNA repair enzymes may be categorized by the type of repair facilitated and include BER (base excision repair) or BER factor enzymes such as uracil-DNA glycosylase (UNG); single strand selective monofunctional uracil DNA glycosylase (SMUG1); 3,N(4)-ethenocytosine glycosylase (MBD4); thymine DNA-glycosylase (TDG); A/G-specific adenine DNA glycosylase (MUTYH); 8-oxoguanine DNA glycosylase (OGG1); endonuclease III-like (NTHL1); 3-methyladenine DNA glycosidase (MPG); DNA glycosylase/AP lyase (NEIL1 or 2); AP endonuclease (APEX 1 and 2), DNA ligase (LIG3), ligase accessory factor (XRCC1); DNA 5′-kinase/3′-phosphatase (PNKP); ADP-ribosyltransferase (PARP1 or 2).

Another category of DNA repair enzymes includes those that are believed to directly reverse damage such as O⁶-MeG alkyl transferase (MGMT); 1-meA dioxygenase (ALKBH2 or ALKBH3).

Yet another category of enzymes operable to repair DNA/protein crosslinks includes Tyr-DNA phosphodiesterase (TDP1).

Also suitable are MMR (mismatch excision repair) DNA repair enzymes such as MutS protein homolog (MSH2); mismatch repair protein (MSH3); mutS homolog 4 (MSH4); MutS homolog 5 (MSH5); or G/T mismatch-binding protein (MSH6); DNA mismatch repair protein (PMS1, PMS2, MLH1, MLH3); Postmeiotic segregation increased 2-like protein (PMS2L3); or postmeiotic segregation increased 2-like 4 pseudogene (PMS2L4).

Also suitable are DNA repair enzymes are those known as nucleotide excision repair (NER) enzymes and include those such as Xeroderma pigmentosum group C-complementing protein (XPC); RAD23 (S. cerevisiae) homolog (RAD23B); caltractin isoform (CETN2); RFA Protein 1, 2, of 3 (RPA1, 2, or 3); 3′ to 5′ DNA helicase (ERCC3); 5′ to 3′ DNA helicase (ERCC2); basic transcription factor (GTF2H1, GTF2H2, GTF2H3, GTF2H4, GTF2H5); CDK activating kinase (CDK7, CCNH); cyclin GI-interacting protein (MNAT1); DNA excision repair protein ERCC-51; excision repair cross-complementing 1 (ERCC1); DNA ligase 1 (LIG1); ATP-dependent helicase (ERCC6); and the like.

Also suitable may be DNA repair enzymes in the category that facilitate homologous recombination and include, but are not limited to DNA repair protein RAD51 homolog (RAD51, RAD51L1, RAD51B etc.); DNA repair protein XRCC2; DNA repair protein XRCC3; DNA repair protein RAD52; ATPase (RAD50); 3′ exonuclease (MRE11A); and so on.

DNA repair enzymes that are DNA polymerases are also suitable and include DNA polymerase beta subunit (POLB); DNA polymerase gamma (POLG); DNA polymerase subunit delta (POLD1); DNA polymerase II subunit A (POLE); DNA polymerase delta auxiliary protein (PCNA); DNA polymerase zeta (POLZ); MAD2 homolog ((REV7); DNA polymerase eta (POLH): DNA polymerase kappa (POLK): and the like.

Various types of DNA repair enzymes that are often referred to as “editing and processing nucleases” include 3′-nuclease; 3′-exonuclease; 5′-exonuclease; endonuclease; and the like.

Other examples of DNA repair enzymes include DNA helicases including such as ATP DNA helicase and so on.

The DNA repair enzymes may be present as components of botanical extracts, bacterial lysates, biological materials, and the like. For example, botanical extracts may contain DNA repair enzymes.

VI. Extracts from Probiotic Microorganisms

Also suitable as adjuvants are extracts from various types of probiotic microorganisms such as Lactobacillus, Bifido bacterium, and the like. One suitable extract is from Lactobacillus and may be in the form of ferment or ferment lysate obtained by fermenting a microorganism from Lactobacillus. Examples of the Lactobacillus genus include, but are not limited to, plantarum, casei, crispatus, etc. The ferment may be in the form of a lysate, filtrate, or both. In the case of a lysate, the fermentation product is lysed. In the case of a filtrate, the fermentation product is filtered. The ingredients may be purchased from Active Concepts under the tradename AC Probiotic 1; Natural F&P Co. Ltd under the tradename Lactobacillus crispatus KLB46; RNA Co. under the trade name K-LAC. The ingredient may also be purchased in the form of mixtures with other ingredients or probiotic organisms. The ferment may be present in amounts ranging from about 0.001 to 10%, preferably from about 0.1 to 5%, more preferably from about 0.1 to 3%.

Another probiotic microorganism may be from Bifido bacterium, and may be in the form of a ferment or ferment lysate from the Bifidobacterium genus. Examples include Bifida ferment extract, Bifida ferment lysate, or Bifida ferment filtrate. The fermentation extract of Bifida may also be in the form of mixtures with other ingredients or probiotic microorganisms. The Bifidobacterium fermentation product may be present in the composition in amounts ranging from about 0.01 to 10%, preferably from about 0.05 to 5%, more preferably from about 0.1 to 2%.

### VII. Other Ingredients

A. Humectants

The composition may contain one or more humectants. If present, they may range from about 0.01 to 75%, preferably from about 0.5 to 70%, more preferably from about 0.5 to 40%. Examples of suitable humectants include glycols, sugars, and the like. Suitable glycols are in monomeric or polymeric form and include polyethylene and polypropylene glycols such as PEG 4-10, which are polyethylene glycols having from 4 to 10 repeating ethylene oxide units; as well as C₁₋₆ alkylene glycols such as propylene glycol, butylene glycol, pentylene glycol, and the like. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, and so on. Also suitable is urea. Preferably, the humectants used in the composition of the invention are C₁₋₆, preferably C₂₋₄ alkylene glycols, most particularly butylene glycol.

B. Surfactants

It may be desirable for the composition to contain one more surfactants, especially if in the emulsion form. However, such surfactants may be used if the compositions are solutions, suspensions, or anhydrous also, and will assist in dispersing ingredients that have polarity, for example pigments. Such surfactants may be silicone or organic based. The surfactants will also aid in the formation of stable emulsions of either the water-in-oil or oil-in-water form. If present, the surfactant may range from about 0.001 to 30%, preferably from about 0.005 to 25%, more preferably from about 0.1 to 20% by weight of the total composition.

1. Organic Nonionic Surfactants

The composition may comprise one or more nonionic organic surfactants. Suitable nonionic surfactants include alkoxylated alcohols or ethers, formed by the reaction of an alcohol with an alkylene oxide, usually ethylene or propylene oxide. Suitable alcohols include mono-, di-, or polyhydric short chain (C1-6) alcohols; aromatic or aliphatic saturated or unsaturated fatty (C12-40) alcohols, of cholesterol; and so on.

In one embodiment the alcohol is cholesterol, or an aromatic or aliphatic saturated or unsaturated fatty alcohol which may have from 6 to 40, preferably from about 10 to 30, more preferably from about 12 to 22 carbon atoms. Examples include oleyl alcohol, cetearyl alcohol, cetyl alcohol, stearyl alcohol, isostearyl alcohol, behenyl alcohol, and the like. Examples of such ingredients include Oleth 2-100; Steareth 2-100; Beheneth 5-30; Ceteareth 2-100; Ceteth 2-100; Choleth 2-100 wherein the number range means the number of repeating ethylene oxide units, e.g. Ceteth 2-100 means Ceteth where the number of repeating ethylene oxide units ranges from 2 to 100. Derivatives of alkoxylated alcohols are also suitable, such as phosphoric acid esters thereof.

Some preferred organic nonionic surfactants include Oleth-3, Oleth-5, Oleth-3 phosphate, Choleth-24; Ceteth-24; and so on.

Also suitable are alkoxylated alcohols formed with mono-, di-, or polyhydric short chain alcohols, for example those having from about 1 to 6 carbon atoms. Examples include glucose, glycerin, or alkylated derivatives thereof. Examples include glycereth 2-100; gluceth 2-100; methyl gluceth 2-100 and so on. More preferred are methyl gluceth-20; glycereth-26 and the like.

Other types of alkoxylated alcohols are suitable surfactants, including ethylene oxide polymers having varying numbers of repeating EO groups, generally referred to as PEG 12 to 200. More preferred are PEG-75, which is may be purchased from Dow Chemical under the trade name Carbowax PEG-3350.

Other suitable nonionic surfactants include alkoxylated sorbitan and alkoxylated sorbitan derivatives. For example, alkoxylation, in particular ethoxylation of sorbitan provides polyalkoxylated sorbitan derivatives. Esterification of polyalkoxylated sorbitan provides sorbitan esters such as the polysorbates. For example, the polyalkyoxylated sorbitan can be esterified with C6-30, preferably C12-22 fatty acids. Examples of such ingredients include Polysorbates 20-85, sorbitan oleate, sorbitan sesquioleate, sorbitan palmitate, sorbitan sesquiisostearate, sorbitan stearate, and so on.

Preferred are topical compositions that contain at least one Mir-146a activator and one or more of: a proteasome activator, a DNA repair enzyme, a CLOCK or PER1 gene activator, an autophagy activator, or combinations thereof.

The topical composition may be applied to the skin one or more times per day. Preferred is where the composition is applied to the skin at night, prior to retiring.

### The Method for Formulating the Topical Composition

### I. The Ingredient

The ingredient that stimulates Mir-146a gene expression can be identified by screening according to the methods set forth herein.

II. Treating Skin Cells In Vitro with the Ingredient

The skin cells may be keratinocytes, fibroblasts, or adipocytes. The skin cells are first collected for in vitro testing. Cells are grown in appropriate culture media, with some of the cells being exposed to varying concentrations of the ingredient that are appropriate for testing.

III. Extracting RNA from the Skin Cells

Treated and untreated cells are then lysed and homogenized by exposing to a solvent that is optimized for isolating RNA from cells. Preferred is a solvent solution containing phenol and a guanidinium compound. The guanidinium compound may be an acid or salt thereof and may include guanidinium thiocyanate or guanidinium hydrochloride in an amount sufficient to protect the RNA components from degradation. Preferred is where the solvent contains from about 0.5 to 2 Molar concentration of guanidinium thiocyanate. The composition may also contain additional thiocyanate compounds such as alkali metal or alkaline earth metal salts such as sodium, potassium, or ammonium, in addition to one or more buffering components such as sodium acetate, sodium citrate, or mixtures thereof in an amount sufficient to maintain the pH of the solution in a range of about 4 to 6. In addition to the phenol, which will extract the proteins from the aqueous phase and inhibit the action of contaminating enzymes that degrade RNA, it may be desirable to include a phenol solubilizer. Suitable phenol stabilizers include polyhydric alcohols like glycerol. Preferred is where the solvent comprising guanidinium thiocyanate at 0.5 to 2 Molar concentration, ammonium thiocyanate at 0.1 to 0.6 Molar, a buffer (e.g. sodium acetate) in an amount to cause the pH of the composition to range from 4 to 6 and glycerol in an amount ranging from 3 to 10% and phenol ranging from 30 to 50%, with all percentages by volume of the total composition. The solvent and process are disclosed in U.S. Pat. No. 5,346,994 which is hereby incorporated by reference in its entirety.

The cells are then sedimented and the RNA present in the aqueous phase is precipitated by treatment with isopropanol and centrifuged to obtain a sediment which is then washed with ethanol and centrifuged again to yield purified total cellular RNA. The amount of total RNA in the sample can be measured by spectrophotometer at a 260 nm wavelength.

IV. Reverse Transcribing the Extracted RNA to Form cDNA Strands

The RNA strands are then reverse transcribed by exposing the RNA sample to reverse transcriptase.

V. Amplifying and Quantifying the cDNA Strands Complementary to Mir-146a with a Primer Sequence Specific for Mir-146a

The cDNA strands are then amplified and quantified by exposing to a tagged primer nucleic acid sequence specific for Mir-146a. This is accomplished by stem loop primers that anneal to the Mir-146a component of the total RNA.

Preferred is where the primer sequence, or probe, has 18-22 nucleotides and is labeled with a fluorophore at the 5′ end and a quencher fluorophore at the 3′ end. The primer is complementary to the Mir-146a nucleotide and may be homologous complementary in whole or in part to the entire Mir-146a nucleotide sequence. The primer sequence, or probe, may be totally or partially homologous to the Mir-146a nucleotide sequence.

The Mir-146a annealed to the probe is then amplified in the PCR mixture to the cDNA complementary to the Mir-146a strand and can be readily quantified by measuring the tagged fluorophore. The data obtained is then compared with the measurements on untreated cells and the control.

VI. Selecting the Ingredient that Shows an Increase in cDNA when Compared to Untreated Cells.

The ingredient that shows an increase in cDNA annealed to the Mir-146a probe compared to untreated or negative control cells is selected for formulating into topical products.

The methods set forth in Bienes, et al., Methods, Volume 50 (2010) pages 244-249 are suitable for use in extract Mir-146a from skin cells to identify ingredients that stimulate Mir-146a activity in skin cells.

### A Method for Treating Skin

The invention is also directed to method for stimulating, in skin cells: (a) Mir-146a expression, (b) CLOCK or PER1 gene expression, (c) cellular DNA repair; and (d) stemness by topically applying a composition where one or more ingredients provide effects (a), (b), (c), and (d) in combination with an adjuvant.

The invention is also directed to a method for treating skin to provide one or more benefits selected from the group: (a) moisturization, (b) reducing skin redness or inflammation, (c) minimizing the appearance of lines and wrinkles, (d) evening skin tone, (e) minimizing the appearance of irregularities and age spots on hands, face, and neck, (f) improving the appearance of uneven pigmentation, (g) or combinations thereof, by topically applying a composition that contains one or more ingredients that: (i) stimulate Mir-146a expression, and (ii) improves repair of damaged cellular DNA, and/or (iii) stimulates CLOCK or PER1 gene activity, and/or (iv) maintains stemness, or expression of stem cell markers that are inherent in stem cells in combination with an adjuvant.

The term “stemness” refers to an essential character of stem cells that differentiate them from other types of cells. Such characteristics include self-renewal and the generation of differentiated progeny. Any ingredient that promotes the normal health and well-being of epidermal stem cells inherently present in an individual's skin is considered to promote stemness. Stemness can be confirmed or quantified by measuring specific markers present on stem cells.

The invention will be further described in connection with the following examples which are set forth for the purposes of illustration only.

### Example 1

An extract of Adansonia digitata (Baobab) enriched in small RNA having 150 nucleotides or less was prepared by combining 50 grams of baobab seed cake in 1 kilogram of distilled water and adding 3.8 grams of tetrasodium EDTA. The pH was adjusted to be between 10.5 and 11. The mixture was stirred for 2 hours at 58° C. and the pH adjusted between 7 to 8. Hydrolysis with a proteolytic enzyme was then carried out by adding 2% by weight of papain to the plant material. The mixture was stirred for 2 hours at 58° C. The extract was centrifuged for 10 minutes at 4000 g to remove the solids. Sequential filtrations were then carried out using filters of decreasing porosity sizes between 20 and 50, and 7-20 microns to clarify the plant extract. The extract was then warmed to 80° C. overnight (for at least 12 hours) to deactivate the enzyme. Additional filtrations were performed with increasingly smaller filters until a porosity of 0.3 to 0.4 millimicrons was achieved. The pH was then readjusted from 6 to 6.5. The extract was then diluted in a mixture of water and 30% glycerol. The resulting extract was a red amber aqueous extract of Baobab and 57 mg/kg small RNA of less than 150 nucleotides in length

### Example 2

Normal human dermal fibroblasts from a 62 year old donor were seeded at a density of 500,000 cells per 60 mm petri dish in Dulbecco's Modified Eagle Medium (“DMEM”) (Life Technology) supplemented with 10% serum and 1% antibiotic.

The following day cells were treated with the following ingredients for 48 hours with 3 independent biological replicates:

(a) Baobab extract at concentrations of 0% (Untreated or Control), 1%, 2% and 3% with samples in three replicates.

(b) Algae Extract (Polysea, PS) at concentrations of 0% (Untreated or Control), 0.2%, 0.5% and 1% of PS active, three independent biological replicates.

(c) Moringa oleifera extract (Nutringa®) at concentrations of 0% (Untreated or Control), 0.0000625%, 0.000125% and 0.00025% of NG active, three independent biological replicates.

(d) Sesame Oil (S.O) at concentrations of 0% (Untreated or Control), 0.01%, 0.05% and 0.1%.

### RNA Extraction:

Total RNA (including microRNAs) was extracted with miRNeasy Micro Kit (50), Cat #217084, QIAGEN. A total of 10 nanograms (ng) of eluted RNA per tube of reaction was utilized to perform reverse transcription (“RT”) with specific RT probes for Mir 146a (target microRNA) and the internal small nuclear RNA (snoRNA) and control RNU44. The RT step was performed with the TaqMan microRNA Assays (Applied Biosystems) as per manufacturer's instructions utilizing the TaqMan microRNA Reverse Transcription Kit, Cat #4366596.

Quantitative Real-Time PCR (“qRT-PCR”) amplification step was performed utilizing a QuantStudio 7 Flex Machine by Applied Biosystems with specific fluorescently labelled probes for Mir-146a and RNU44. Real time gene expression levels of treated cells were measured against untreated cells and controls. Quantification of the gene target was achieved by analyzing the cycle threshold (Ct) values, that is, the number of cycles required for the fluorescent signal to cross the threshold or exceed the background level. The resulting Ct threshold cycle values of the samples treated with the test extracts were normalized to the Ct untreated control. Gene expression change values plotted on the y axis vs the concentrations of the ingredient plotted on the x axis were graphed in Graph Prism 5 Software, San Diego, Calif. The same procedure was used for the other test samples.

The rest of the eluted RNA was amplified with random primers using the cDNA High Capacity Reverse Transcription Kit Cat #43668814 and quantitative Real-Time PCR (qRT-PCR) amplification step was performed utilizing QuantStudio 7 Flex Machine from Applied Biosystems with specific fluorescently labelled probes for GAPDH (internal control), (PER1 and Lin28A as target messenger RNA (mRNAs). Quantification of gene target was achieved by analyzing the Ct values. Analyzed data where PCR cycle expression change values (plotted on the y axis) vs the concentrations (e.g. 1%, 2% and 3%) of the active (plotted on the x axis) were graphed in Graph Prism 5 Software, San Diego, Calif.

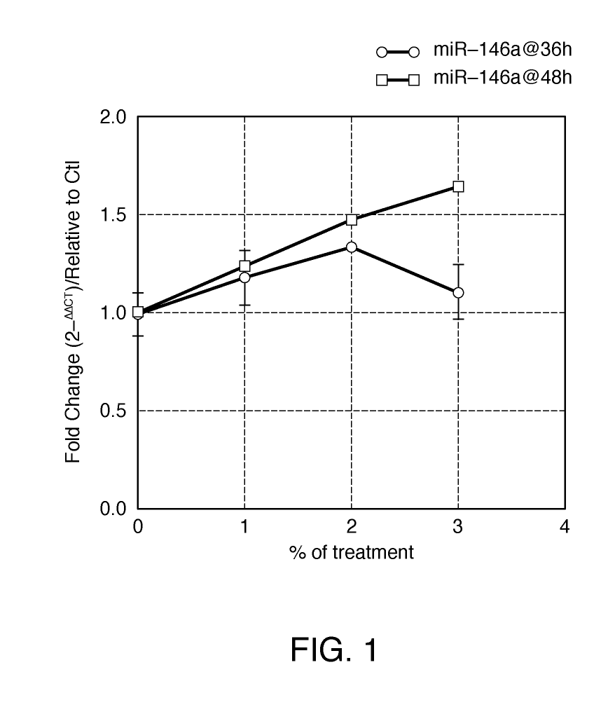

The results for Acorus calamus extract are set forth in FIG. 1.

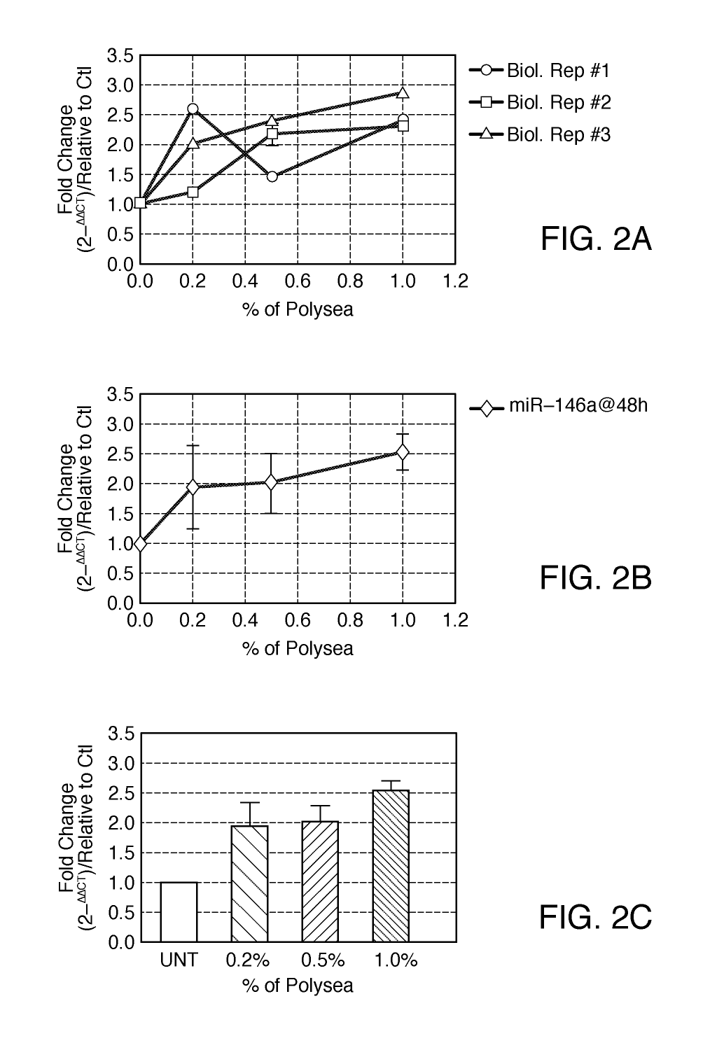

The results for Algae Extract (Polysea) are set forth in FIG. 2

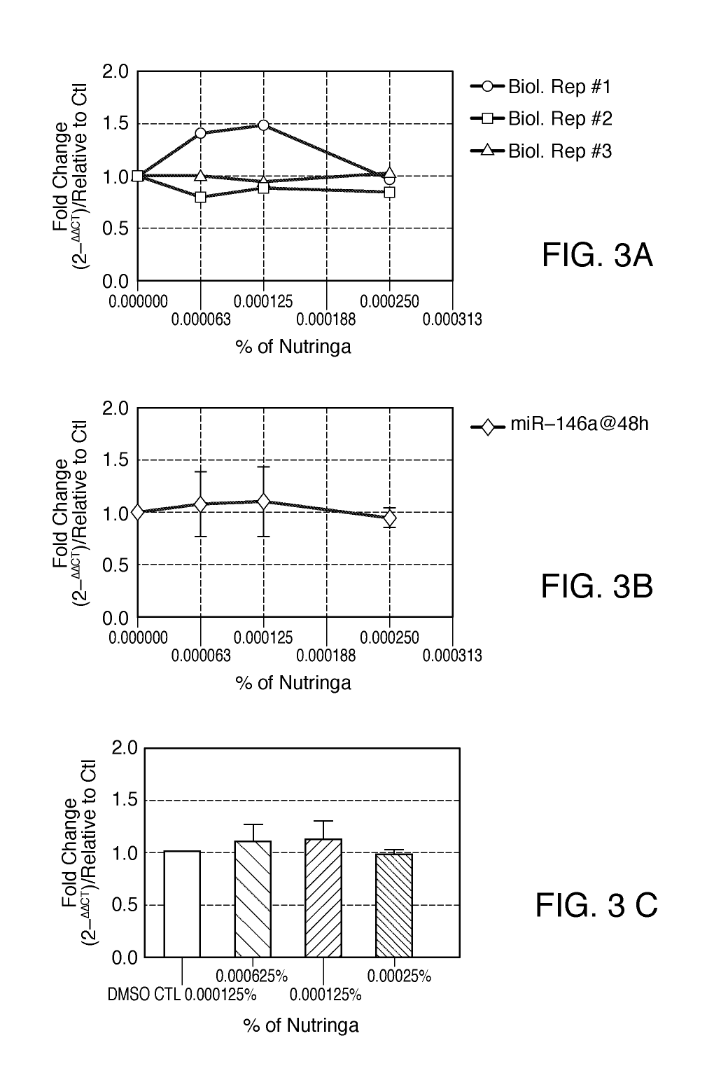

The results for Moringa oleifera extract are set forth in FIG. 3.

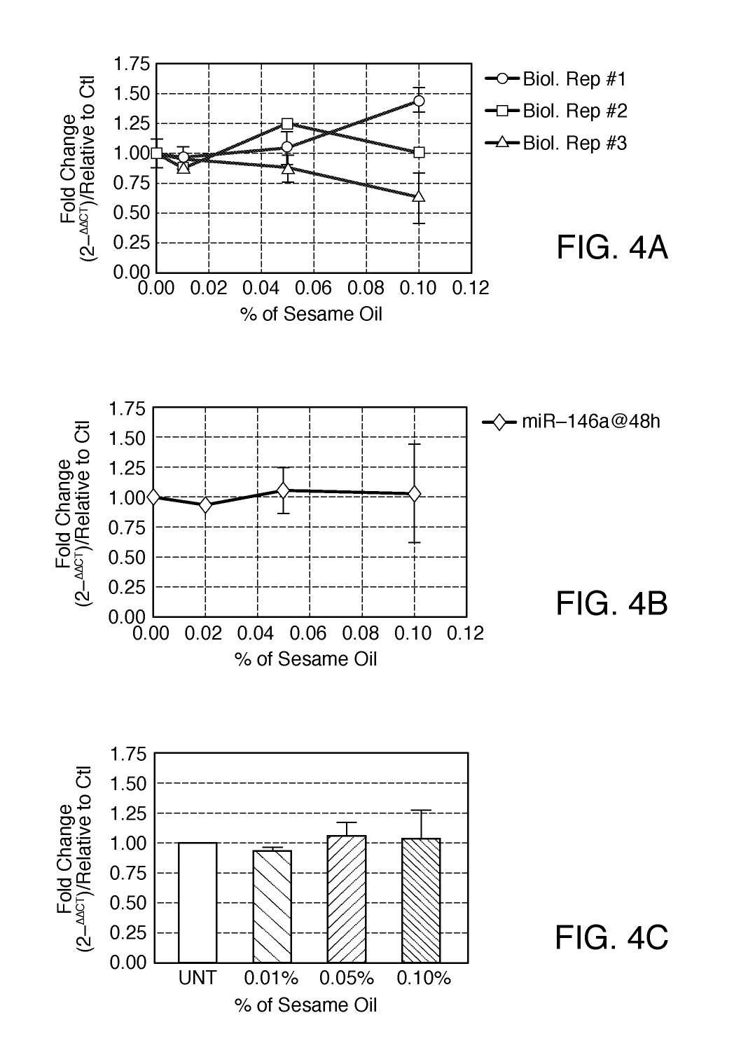

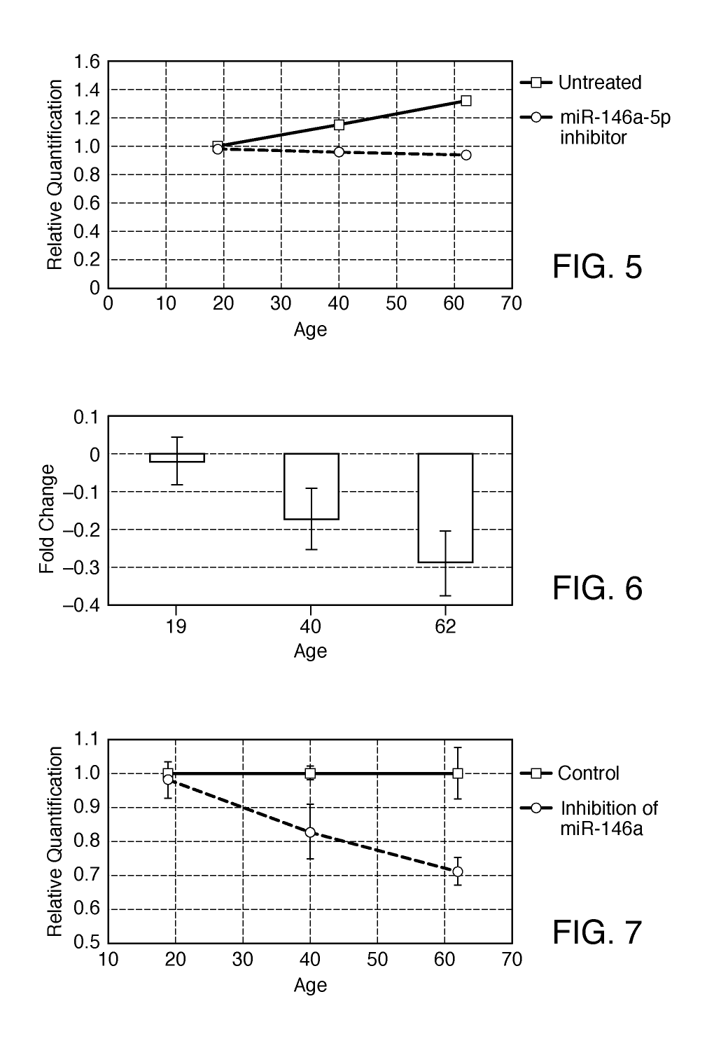

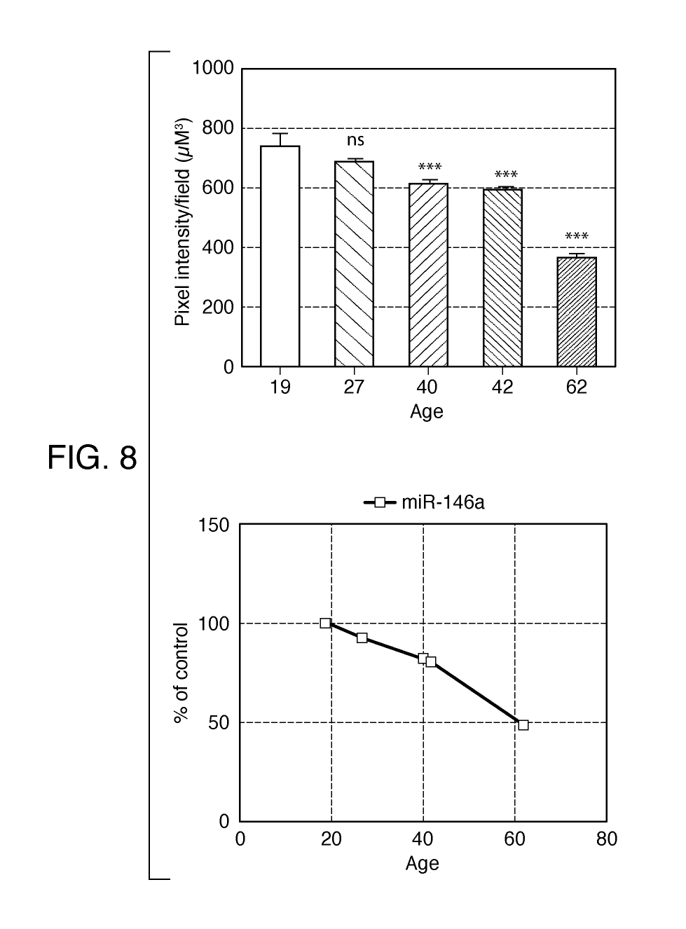

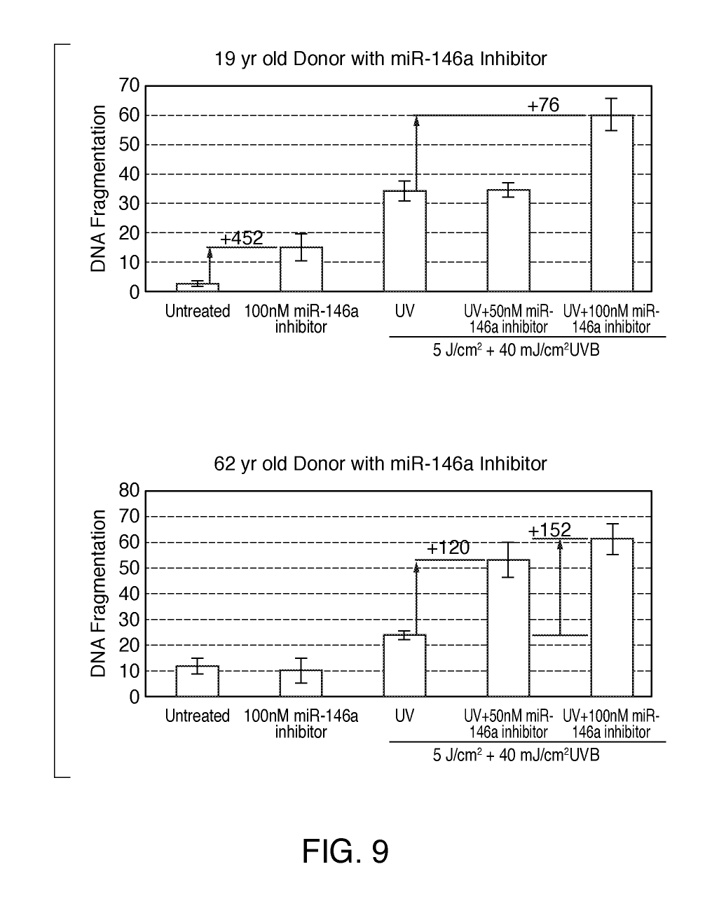

The results for sesame oil are set forth in FIG. 4.

The results demonstrate that Acorus calamus extract and Algae Extract stimulated Mir-146a gene expression in test cells while Moringa oleifera and sesame oil were not effective.

### Example 3

Various ingredients were tested for Mir-146a and to study the relationship between Mir-146a and PER1 activation.

There was an age dependent increase in PER1 expression with age in the untreated cells. Inhibition of miR-146a lowered PER1 expression in all aged cells, but had the greatest impact on the 62y old cells and the smallest effect on cells from the 19 year old donor.

In this experiment miR-146a was inhibited in fibroblasts from 3 different aged donors to see how it affected PER1 expression.

Normal Human Dermal Fibroblasts from 19, 40 & 62 year old donors p6, p6 & p9
DMEM (Life Technologies, cat#11965-092)
Bovine Calf Serum (Thermo, cat#SH30072.03)
Penicillin Streptomycin (Cellgro, cat#30-001-CI)
DPBS (Corning, cat#21-031-CV)
hsa-miR-146 mirVana Inhibitor (Ambion, cat#4464084)
Dharmafect 3 Transfection Reagent (GE Life Sciences, cat#T-2003-01)
Quant-iT™ RiboGreen® RNA Assay Kit (Life Technologies, cat#R11490)
High Capacity cDNA Archive Kit (Life Technologies, cat#7322171)
TaqMan Fast Universal PCR Master Mix (Life Technologies, cat#4352042)
RNase Inhibitor (Life Technologies, cat#N808-0119)
GAPDH primer pair (Life Technologies, cat# Hs10192604_m1)
Perl primer pair (Life Technologies, cat#Hs00797944_s1)
MicroAmp Fast Optical 96-well Reaction Plate (Life Technologies, cat#4346906)
MicroAmp Optical Adhesive Film (Life Technologies, cat#4311971)

### Methods:

Cells were plated in 6 cm plates at 400,000 cells per plate and incubated for 24 hours.
The inhibitor was resuspended 100 μl H₂O to make a 50 mM solution
100 μM Inhibitor (total volume 18.2 mL) was prepared
Two tubes were prepared and incubated at room temperature for 5 minutes.

| | Tube 1 | Tube 2 |
|---|---|---|
| 50 μM Inhibitor | 36.4 μl | — |
| Serum-free media (DMEM only) | 1783.6 μl | 1747.2 μl |
| Dharmafect #3 | — | 72.8 μl |

The contents of both tubes were combined in a 50 mL conical tube and incubate for 20 minutes at room temperature (25° C.). 14.56 mL Penicillin/Streptomycin free media (Dulbecco's Modified Eagle Medium (DMEM)+10% Bovine calf serum without penicillin/streptomycin) was added.
Cells were washed once with PBS, followed by 2 mL of the treatment ingredients for 24 hours.
RNA was extracted and quantified as set forth in Example 1.

The results are set forth in FIG. 4 and show that when Mir-146a activity was inhibited in cells the PER1 expression was also inhibited, thus showing a positive correlation between Mir-146a activity and PER1 expression.

Inhibition of miR-146a decreased PER1 expression in cells from all aged donors having the greatest effect on the cells from the 62y old donor. The effect of inhibition on Perl expression on the cells from the 19y old donor was minimal.

### Example 4

Polysea was screened for Mir-146a activation by plating cells into a 35 mm glass bottom cell culture dish at an approximate concentration of 11,000 cells/dish. The cells were suspended in 200 μl of Media (Dulbecco's Modified Eagle Medium (Thermo Fisher Scientific, cat#11965-092)+1% Penicillin/Streptomycin (Corning, cat#30-001-CI)+10% Bovine Calf Serum (HyClone, cat#SH30072.03)) and applied evenly to the depression in the center of the dish. After 1 hour 3 ml. of Media was added to the dish. The cells were incubated at 37° C., 5% CO₂, and 95% humidity for 24 hours at 37° C. SmartFlare® probes for Mir-146a (EMD Millipore, cat#SF-500) were reconstituted with 50 μl sterile H₂O directly to the vial. The vials were stored. SmartFlare® solution was then prepared by combining 10 μl of the reconstituted probe and 10 ml Media and the vial inverted to mix. SmartFlare® solution, 2 ml, was placed into each dish and the dish was incubated for 16 hours. The cells were washed with Dulbecco's Phosphate Buffered Saline (DPBS, Corning, cat#21-031-CV), followed by incubation for 16 hours. A 1:1000 solution of Hoechst 33342 Nuclear Stain (Enzo, cat#ENZ-51031-HOE33342), was prepared by combining 10 μl solution with and 10 ml Media. 2 ml of the 1:1000 Hoechst solution was placed into each dish, and the dishes were incubated for 5 min at room temperature. Cells were washed with DPBS. Into each dish 1 ml of media was placed. Images were captured using a Nikon A1 confocal microscope with Nikon Elements. Settings for 20× were as follows:

### Control by: Frame/sec ¼

### Size 1024

### Normal

### Ch Series

### Pinhole 1.2 AU

### Channel Settings: DAPI/HV100/Offset 0/Laser 5

### FITC: None

### TRITC: HV145/Offset 0/Laser 8

### Cy5.5: HV120/Offset 0/Laser 5

The volume measurement tool in the Nikon Elements Software was used to measure the amount of Mir-146a via fluorescence in the images.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

1. A topical composition containing an adjuvant and at least one ingredient that stimulates Mir-146a expression in skin cells.

2. The composition of claim 1 wherein the adjuvant is selected from the group consisting of (a) DNA repair enzyme, (b) PER1 gene expression activator, (c) CLOCK gene expression activator, (d) autophagy activator, (e) proteasome activator, (f) an extract from Lactobacillus genus, (g) an extract from Bifidobacterium genus; and (h) mixtures thereof.

3. The composition of claim 2 wherein the extract from Bifidobacterium genus is a ferment, filtrate, or lysate from Bifidobacterium.

4. The composition of claim 2 wherein the extract from Lactobacillus is a lysate, filtrate, or ferment.

5. The composition of claim 2 wherein the autophagy activator is selected from the group consisting of extracts from the genera Lithothamnium, Melilot, Citrus, Candida, Lens, Urtica, Carambola, Momordica, Yarrowia, Plumbago, or combinations thereof.

6. The composition of claim 5 wherein the autophagy activator is an extract from Candida genus which is Candida saitoana.

7. The composition of claim 2 wherein the proteasome activator is selected from the group consisting of algin, alginates, hydrolyzed algin, molasses extract, Trametes extracts, and mixtures thereof.

8. The composition of claim 2 wherein the PER1 gene expression activator is selected from the group consisting of Tripeptide-32, Tetrapeptide-26, and mixtures thereof.

9. A method for formulating a topical composition containing an adjuvant and at least one ingredient that stimulates expression of Mir-146a activity in skin cells comprising the steps of:
(a) treating skin cells in vitro with an ingredient,
(b) extracting RNA from skin cells,
(c) reverse transcribing RNA to form cDNA strands,
(d) amplifying and quantifying the cDNA strands complementary to Mir-146a by probing with a primer sequence and annealing cDNA strands complementary to Mir-146a,
(e) selecting the ingredient that shows an increase in cDNA complementary to Mir-146a when compared to untreated cells.

10. The method of claim 9 wherein the skin cells are keratinocytes or fibroblasts.

11. A method for stimulating, in skin cells: (a) Mir-146a expression, (b) CLOCK or PER1 gene expression, (c) cellular DNA repair; and (d) stemness by topically applying a topical composition containing at least one adjuvant and an ingredient that activates (a), (b), (c), and (d).

12. The method of claim 11 wherein the adjuvant is a ferment, filtrate, or lysate of Bifidobacterium and (a) is Adansonia digitata extract, (b) is Tripeptide-32, and (c) is a DNA repair enzyme.

13. The method of claim 11 wherein the adjuvant is a ferment, filtrate, or lysate of Lactobacillus and (a) is Adansonia digitata extract, (b) is Tripeptide-32, and (c) is a DNA repair enzyme.

14. A method for treating skin to provide one or more benefits selected from the group: (a) moisturization, (b) reducing skin redness or inflammation, (c) minimizing the appearance of lines and wrinkles, (d) evening skin tone, (e) minimizing the appearance of irregularities and age spots on hands, face, and neck, (f) improving the appearance of uneven pigmentation, (g) repairing skin that is damaged by exposure to sunlight, pollution, environmental conditions, and the like, (h) improving skin laxity, or combinations thereof, by topically applying a composition that contains an adjuvant and one or more ingredients that: (i) stimulate Mir-146a expression, and (ii) improve repair of damaged cellular DNA, and/or (iii) stimulate CLOCK or PER1 gene activity, and/or (iv) promote stemness by maintaining expression of stem cell markers that are inherent in stem cells and an adjuvant.

15. The method of claim 14 wherein the benefit is promoting stemness.
